(19) United States
(12) Patent Application Publication
(10) Pub. No.: US 2011/0305642 A1
(43) Pub. Date: Dec. 15, 2011

(54) MICRONUTRIENT SUPPLEMENT

(75) Inventors: Éric Gervais, Laval (CA); Gordana Atanackovic, Toronto (CA); Pierre Boivin, Laval (CA)

(73) Assignee: DUCHESNAY INC., Laval (CA)

(21) Appl. No.: 13/214,644

(22) Filed: Aug. 22, 2011

Related U.S. Application Data
(63) Continuation of application No. 10/922,799, filed on Aug. 20, 2004.

(30) Foreign Application Priority Data
Aug. 21, 2003 (CA) 2438043
Aug. 21, 2003 (CA) 2438155

(51) Int. Cl. A61K 33/26; A61K 9/00; A61P 3/02; A61K 33/34

(52) U.S. Cl. 424/10.3; 424/638; 424/646

(57) ABSTRACT

A micronutrient supplement the supplement being characterized by having at least two types of distinct dosage units wherein said distinct dosage units which physically separate nutritional, vitamin or mineral supplements which are known or proven to be negatively interacting when co-mingled or co-administered, the distinct dosage units being designed to be taken at a predetermined time interval.

This application claims priority to co-pending Canadian Application No. 2,438,043 filed Aug. 21, 2003 and Canadian Application No. 2,438,155 filed Aug. 21, 2003. The entire text of the above applications are incorporated by reference.

### FIELD OF THE INVENTION

The present invention relates to a micronutrient supplement containing ingredients such as multivitamins, minerals, fatty acids, amino acids, plant extracts, and the like.

### BACKGROUND OF THE INVENTION

Micronutrient compositions are commonly taken as dietary aids; either as therapeutic preparations directed to a specific medical problem or as general nutritional supplements. Micronutrients may be broadly defined as substances that are essential or helpful for the maintenance of normal or enhanced metabolic function, but are not normally or sufficiently synthesized in the body and must thus be supplied from an exogenous source.

Given poor dietary habits of individuals and other factors, it has become clear that the role of micronutrient compositions is substantial when it comes to preventing fatigue, disease and optimizing cell maintenance and development. This is particularly the case for individuals who lead a stressful lifestyle, for pregnant women or those who engage in a large amount of physical exercise. Additionally, many drugs, some chronic diseases (e.g. rheumatoid arthritis), certain cancer treatments, and alcoholism can all lead to a deficiency in one or more micronutrients.

It is has also been suggested that a significant portion of preventable illnesses (which it is estimated absorbs as much as 70 per cent of total health care costs in the United States) could be readily prevented through supplementing the diet with micronutrients. In addition to major health care cost savings other benefits of supplementation include better quality of life, longer life, and increased productivity. The level of supplements required for effective disease protection cannot be obtained through even the most healthful diet (Bendich, Adrianne, et al. Potential health economic benefits of vitamin supplementation. Western Journal of Medicine, Vol. 166, May 1997, pp. 306-12).

Micronutrients are especially important to pregnant or lactating women, ensuring an adequate provision of nutrients for the developing fetus and for the mother. It has become clear that the role of micronutrients is substantial when it comes to preventing fatigue, disease and optimizing cell maintenance and development.

Many micronutrient supplements however, pose a potential problem of nutrient-nutrient interactions. The presence or excess of one nutrient in such a supplement may interact with another nutrient, thereby adversely affecting its absorption. Iron for example is reported to inhibit the co-absorption of zinc and vice-versa (Hambridge et al., Obstet. Gynecol. 4:593-596, 1987); zinc is reported to inhibit the co-absorption of copper (Festa et al., Am. J. Clin. Nutr. 41:285-292, 1985); calcium is reported to interfere with the co-absorption of both iron and zinc (Seligman et al., Obstet. Gynecol. 61:356-362, 1983); and protein supplements are reported to increase urinary calcium losses (Allen et al., Am. J. Clin. Nutr. 32: 741-749. 1979) and to increase vitamin B₆ requirements (National Research Council, Recommended Dietary Allowances, 10^{th} ed., Natl. Acad. Press, Washington, D.C. 1989). Iron and copper are also known to degrade folic acid and vitamin B12 when commingled.

Prior art efforts at multivitamins and nutritional aids have often circumvented this problem by using greater doses of ill-absorbed or degraded nutrients. Indeed, there has been a trend towards using mega doses of many nutrients. Such practice is a risky one since an overdose of many nutrients, in particular metallic compounds, can be toxic, thereby achieving the opposite result of creating sickness rather than preventing it. One notable example is iron.

Iron-deficiency anemia is a primary risk during pregnancy because of the increasing red blood cell mass of the mother, the demands of the fetus and placenta (more so in the second and third trimesters of pregnancy), and blood losses during childbirth. Thus, prevention of iron deficiency is of prime importance. A common problem with prior art supplements is that little of the iron ingredients is actually absorbed in the blood stream. The known way to deal with this is to use larger doses of iron ingredients which in turn triggers constipation, nausea when taken on an empty stomach and a metallic taste (Solvell L.; Oral iron therapy: Side effects. In Iron Deficiency: Pathogenesis, Clinical Aspects, Therapy Edited by L Hallberg, H G Harwerth and A Vannotti: London, Academic Press, 1970, pp. 573-583).

Another important micronutrient is folic acid. Studies have revealed that folic acid may play an important role in preventing some types of cancers (e.g. Stolzenberg-Solomon, Rachael Z., et al. Dietary and other methyl-group availability factors and pancreatic cancer risk in a cohort of male smokers. American Journal of Epidemiology, Vol. 153, Apr. 1, 2001, pp. 680-87), heart disease (Loria, Catherine M., et al. Serum folate and cardiovascular disease mortality among US men and women. Archives of Internal Medicine, Vol. 160, November 27, 2000, pp. 3258-62.), and depression (Alpert, Jonathan E. and Fava, Maurizio. Nutrition and depression: the role of folate. Nutrition Reviews, Vol. 55, May 1997, pp. 145-49). It is also well established that taking folic acid before and during pregnancy as a nutritional supplement greatly reduces risks of fetal diseases such as spina-bifida or cleft lip and palate. If use of the supplement containing folic acid is discontinued because of iron intolerance, the benefits of the folic acid will be lost.

Thus, the importance of many of the ingredients present in micronutrient supplements may not be overstated.

In the prior art, U.S. Pat. No. 5,932,624, discloses vitamin supplements comprising folic acid and vitamin B₁₂, and which are essentially free of antioxidants such as phytochemicals, certain vitamins, and minerals such as iron and copper, which are known to destroy some of the vitamin B₁₂ and folic acid. However, such vitamin supplement, in an effort to avoid co-absorption problems provides an incomplete product, which fails to include important components such as iron and copper.

U.S. Pat. No. 5,976,568 provides examples of various multivitamins some of them to be taken twice a day. However, the ingredients of the morning and evening tablets are identical. The apparent purpose of the twice-a-day formulation is to provide a second dose of ingredients, which may have been used up during the day. Another drawback of the multivitamin compositions proposed in this prior art is the presence of many competing nutrients in a single dosage unit, e.g. iron, calcium and zinc.

Other problems often associated with patient non-compliance with recommended use of multivitamins and nutritional supplements are related to the large size of the tablets. For example, in the case of pregnant women, because pregnant women are sometimes nauseated and because of their normal instinct to avoid pharmaceutical products, the size and appearance of current products is often enough to cause a pregnant woman to discontinue taking the multivitamin or nutritional aid.

Published Canadian patent application No. 2,258,868 discloses an attempt as creating a slightly smaller tablet. The tablet composition is said to provide high levels of calcium (calcium citrate) and iron (carbonyl iron) while maintaining a smaller than usual size. Calcium citrate is described as enhancing the absorption of iron, zinc and magnesium, and as being more soluble, better absorbed and better tolerated than traditional calcium supplements. Carbonyl iron on the other hand, is described as having a higher iron content as compared to the ferrous salts. The use of Ultradense™ calcium citrate and carbonyl iron allows the formulation to be compressed into acceptably sized tablets.

Canadian patent application No. 2,144,751 and U.S. Pat. No. 5,494,678, discloses a multivitamin and mineral supplement for pregnant women. The iron component is said to be ferrous sulfate, coated with a pharmaceutically acceptable film forming material. The coating is said to provide for the release of the ferrous sulfate in the intestine, thus apparently minimizing interactions between iron and divalent cations such as calcium (also in the supplement), in turn improving the iron bioavailability. Thus, the co-absorption problem is dealt with by suggesting that absorption of various components should be engineered to occur at different body sites.

U.S. Pat. No. 4,431,634 discloses multi-mineral prenatal dietary supplements, said to maximize the bioavailability of iron. This is apparently accomplished by maintaining the amount of calcium compounds in the supplement at 300 mg or less, and the amount of magnesium compounds at 75 mg or less, per dosage unit.

Despite the foregoing efforts to improve micronutrient supplements, there remains a need to develop micronutrient compositions overcoming the drawbacks of prior art compositions.

The micronutrient supplement of the present invention seeks to avoid deleterious co-absorption problems associated with co-mingled ingredients.

The micronutrient supplement of the present invention also seeks to provide rather small and palatable dosage units when compared to those of the prior art.

In a preferred embodiment, the micronutrient supplement of the present invention provides optimal nutritional components and amounts that have been found to benefit both fetal growth and the mother's health throughout the pregnancy.

The micronutrient supplement of the present invention also allows the presentation of the tablet ingredients in the form of a plurality of different dosage units so that a patient can voluntarily take some ingredients and not others in case they suffer from intolerance or side effects caused by specific ingredients such as iron.

The present invention seeks to meet these and other needs.

The words “a” and “an,” as used in this specification, including the claims, denote “one or more.” Specifically, the use of “comprising,” “having,” or other open language in claims that claim a combination or method employing “an object,” denotes that “one or more of the object” may be employed in the claimed method or combination.

### SUMMARY OF THE INVENTION

In general terms, the present invention provides a micronutrient supplement, the supplement being characterized by having at least two types of dosage units designed to be taken at a predetermined time interval.

In a preferred embodiment, micronutrients such as vitamin and mineral supplements which are known or proven to be absorption-competing when co-administered are thus be prepared as separate and distinct dosage units and are administered at spaced time intervals so as to minimize drop-offs in absorption and co-absorption problems.

Still in a preferred embodiment, micronutrients such as vitamin and mineral supplements, which are known to potentially cause deleterious side effects, for example constipation in the case of iron supplements, can be grouped in a separate and distinct dosage unit. Therefore, by virtue of the present invention a patient may temporarily stop taking a type of dosage unit of the invention and continue to take the other dosage unit(s) of the invention. The overall effect is to avoid discontinuing the use of supplements entirely and thereby avoiding discontinuance of important ingredients unrelated to the side effects of some ingredients.

Still in a preferred embodiment, the micronutrient supplement is destined for pregnant women and provides optional nutritional components and amounts that have been found to benefit both fetal growth and the mother's health before, throughout and after pregnancy (post-partum).

The present invention also provides a micronutrient supplement in the form of a kit comprising a plurality of types of dosage units along with instructions for taking the dosage units at spaced time intervals.

### BRIEF DESCRIPTION OF THE DRAWINGS

Having thus generally described the invention, reference will now be made to the accompanying drawings, showing by way of illustration a preferred embodiment thereof, and in which:

FIG. 1 shows a perspective view of an example of a kit of the present invention and more specifically an individual blister pack of a week's worth of the supplement of the present invention having an array of a first type of dosage unit to be taken at a given time of day and an array of a second type of dosage unit to be taken at another time of day.

### DESCRIPTION OF THE PREFERRED EMBODIMENT

The present invention will now be described by means of an illustrative and preferred embodiment.

In a most preferred embodiment, the invention discloses a micronutrient supplement in the form of two distinct dosage units to be taken at spaced time intervals. Ideally, the time interval will be 12 hours, however, the time interval may be as short as 4 hours.

The two distinct dosage units and the time interval recommended between ingestion of the distinct dosage units will of course be the domain of those of skill in the art and may afford variations. The two distinct dosage units and recommended time intervals between ingestion is primarily aimed at minimizing known or eventual vitamin-vitamin, vitamin-mineral and mineral-mineral deleterious interactions.

An added benefit of the two distinct dosage units is the possibility for a patient to discontinue taking the type of dosage unit causing unwanted side effects while continuing with the other type of dosage unit.

An added benefit of the two distinct dosage units is the possibility for the manufacturer to produce a smaller and more palatable dosage unit, which improves patient compliance when compared to unpalatable large dosage units. The possibility for the manufacturer to produce smaller units is not only related to the fractionating of a daily dose into two dosage units but also because of the reduction of ingredient interactions which caused manufacturers to use greater quantities of ill-absorbed ingredients.

More specifically, in the most preferred embodiment of the invention, the calcium and iron ingredients are placed in distinct and different dosage units so as to avoid their known propensity to mutually interfere with their absorption.

Also in a most preferred embodiment, the folic acid and iron ingredients are placed in distinct and different dosage units so as to allow discontinuance of the iron-containing dosage unit while maintaining ingestion of the folic acid-containing dosage unit.

Also in a most preferred embodiment, the amount of zinc present in the micronutrient supplement of the present invention, has been reduced in order to further improve the iron bioavailability.

Furthermore, the presently disclosed micronutrient supplement comprises a greater iron/vitamin C ratio (1:3.4), further improving iron bioavailability.

In a most preferred and convenient embodiment, the micronutrient supplement of the present invention will be provided with instructions to take a first type of dosage unit in the morning and a second type of dosage unit in the evening. In a most preferred embodiment, the folic acid ingredient will be present in the evening dosage unit while the iron ingredient will be present in the morning dosage unit. In cases where a patient suffers from constipation as a result of the iron supplement, the patient would be able to halt the morning unit while continuing to take the important evening unit comprising folic acid. It is indeed of interest that patients have enough folic acid in their bodies given its potential prophylactic affect against certain types of cancers, heart disease, depression and for preventing certain types of birth defects.

Thus the present invention provides a micronutrient supplement formulation having distinct dosage units to be taken at spaced apart time intervals. Most conveniently, one type of dosage unit can be taken in the morning and the second type in the evening. Hence, the AM and PM formulations contain different ingredients. Each set of ingredients is aimed at providing optimal nutritional components and amounts, while concurrently minimizing the undesired problems of the conventional unitary formulations.

In a preferred embodiment, the micronutrient supplement of the present invention will feature an AM dosage unit composition comprising: provitamin A (beta-carotene), vitamin E (di-α-tocopheryl acetate), vitamin C (ascorbic acid), vitamin B₁ (thiamine mononitrate), vitamin B₂ (riboflavin), vitamin B₃ (niacinamide), vitamin B₆ (pyridoxine HCl), vitamin B₅ pantothenic acid (calcium pantothenate), magnesium (magnesium oxide), iodine (potassium iodide), iron (ferrous fumarate), copper (cupric oxide), zinc (zinc oxide), and pharmaceutically acceptable excipients;

and the PM dosage unit composition will comprise: vitamin D₃ (cholecalciferol), vitamin B₁₂ (cyanocobalamin), folic acid, calcium (calcium carbonate) and pharmaceutically acceptable excipients.

Description of Various Preferred Ingredients in Best Mode

When referring to quantities of preferred ingredients reference is made to quantities of pure substance regardless of form. For example, when referring to quantities of calcium or iron, reference is made to elemental calcium and elemental iron as opposed to quantities of calcium carbonate and ferrous fumarate. It is to be understood that adequate quantities of calcium carbonate and ferrous fumarate would be used to contain the chosen amount of elemental calcium or iron.

Throughout this disclosure, when referring to the term about, was is to be understood is a variation of plus or minus 20% wt.

Beta-carotene or provitamin A is a precursor to vitamin A. Beta carotene is a potent antioxidant that appears to work synergistically with several vitamins, minerals and antioxidants. Beta-carotene is provided in the present micronutrient formulation in amounts of about 250 to 5000 I.U.; and most preferably about 2700 I.U.

Vitamin B₁ (thiamine mononitrate) is an essential water-soluble B-vitamin playing an important role in the metabolism of carbohydrates. It is critical for the transmission of high-frequency impulses in the central nervous system. Vitamin B₁ is provided in the present micronutrient formulation in amounts of about 0.5 to 10 mg; most preferably about 3.0 mg.

Vitamin B₂ (riboflavin) is an essential water-soluble B-vitamin that is required for the repair and growth of tissues as well as for DNA synthesis. It also assists in the metabolism of nutrients. Vitamin B₂ is provided in the present micronutrient formulation in amounts of about 0.5 to 10 mg and most preferably about 3.4 mg.

Vitamin B₃ (niacinamide) is the amide form of the vitamin Niacine, and is an essential constituent of coenzymes I and II, occurring in a wide variety of enzyme systems, and which are involved in the anaerobic oxidation of carbohydrates. Vitamin B₃ is provided in the present micronutrient formulation in amounts of about 2 to 50 mg and most preferably about 20.0 mg.

Vitamin B₆ (Pyridoxine HCl) is a term commonly used for a group of vitamins consisting of pyridoxine, pyridoxal, pyridoxal-5-phosphate, pyridoxamine, and pyridoxamine-5-phosphate. These vitamins are important in protein and amino acid metabolism and are required to synthesize hemoglobin. Vitamin B₆ is provided in the present micronutrient formulation in amounts of about 2 to 100 mg and most preferably about 10.0 mg.

Vitamin B₁₂ (cyanocobalamine) is an essential water-soluble B vitamin that is provided in the present micronutrient formulation in amounts of about 2 to 50 mcg and most preferably about 12.0 mcg.

Folic acid is a water soluble B-vitamin that helps build healthy cells. Folic acid is necessary for the synthesis of RNA and DNA. Folic acid is provided in the present micronutrient formulation in amounts of about 0.1 to 10 mg and most preferably about 1.1 mg. Since folic acid is water soluble, it is readily eliminated from the body, and therefore has to be taken daily to help prevent, for example, neural tube defects in the fetus. During periods of rapid growth, such as during pregnancy and fetal development, the body's requirement for this vitamin increases. Patients having enough folic acid in their bodies can decrease the risk of some types of cancers, heart disease and even depression. The U.S. Public Health Service currently recommends 400 micrograms of folic acid every day.

Vitamin B₅ Pantothenic acid (calcium pantothenate) is a water-soluble vitamin that plays an active role in the metabolism of proteins, fats and carbohydrates. It is also involved in the synthesis of sterols, hormones, porphyrins and acetylcholine. Pantothenic acid is provided in the present micronutrient formulation in amounts of about 0.5 to 20 mg and most preferably about 5.0 mg.

Pharmaceutically acceptable forms of certain of the B vitamins include, but are not limited to, thiamine mononitrate or thiamine hydrochloride, niacin or niacinamide; and pyridoxine hydrochloride.

Vitamin C (ascorbic acid) is an essential water-soluble vitamin that functions as an antioxidant. It is critical in producing and maintaining collagen and promotes wound healing. It is also important in producing hormones that regulate basal metabolic rate and body temperature. Vitamin C (ascorbic acid) is provided in the present micronutrient formulation in amounts of about 10 to 1000 mg and most preferably about 120.0 mg. Pharmaceutically acceptable salts of ascorbic acid include, but are not limited to sodium or calcium ascorbate.

Vitamin D₃ (cholecalciferol) is an essential fat-soluble vitamin whose major biological function is to maintain normal blood levels of calcium and phosphorus. Vitamin D₃ is provided in the present micronutrient formulation in amounts of about 10 to 1000 I.U. and most preferably about 250.0 I.U. vitamin D₃ (cholecalciferol). The vitamin D₃ used in the present formulation can include any of the forms of vitamin D that is a precursor to cholecalciferol.

Vitamin E (dl-α-tocopheryl acetate) is a fat-soluble vitamin functioning as an antioxidant protecting lipid membranes from oxidation. Vitamin E (dl-α-tocopheryl acetate) is provided in the present micronutrient formulation in amounts of about 1 to 500 I.U. and most preferably about 30 I.U. Vitamin E can also be present as α, β-, γ-, or δ-tocopheryl, or as a mixture or as an isomer thereof, such as dl-α-tocopheryl acetate or α-tocopheryl acetate. Salts of vitamin E include, but are not limited to, an acetate, or acid succinate salt.

Calcium (calcium carbonate) is required for adequate bone formation and maintenance, as well as for diverse metabolic functions. Calcium is involved in the transmission of nerve impulses, muscle contraction and relaxation, blood clotting, structure and function of cell membranes and vitamin B₁₂ absorption. Women are advised to increase their calcium intake substantially during pregnancy. Calcium is provided in the present micronutrient formulation in amounts of about 10 to 1500 mg and most preferably about 300.0 mg, in the form of suitable amounts of calcium carbonate to equate to the required amount of calcium. Calcium carbonate relies on stomach acid to dissolve. Supplemental calcium is beneficial for the skeletal system.

Iron (ferrous fumarate) is an essential mineral playing an important role in the transport of oxygen to tissues throughout the body via hemoglobin and myoglobin. Iron is provided in the present micronutrient formulation in the form of ferrous fumarate, corresponding to amounts of elemental iron of about 2 to 300 mg and most preferably about 35 mg.

Magnesium (magnesium oxide) is an essential mineral for many biological processes. Magnesium is provided in the present micronutrient formulation in the form of magnesium oxide, in amounts of about 5 to 200 mg and most preferably about 50 mg. Magnesium can be incorporated in the present micronutrient formulation in various forms such as an oxide, a sulfate, or the like.

Zinc (zinc oxide) is a trace mineral essential to cell multiplication, tissue regeneration and wound healing. It is required in many enzymatic functions throughout the body, and also helps regulate the immune system and insulin metabolism. Zinc is provided in the present micronutrient formulation in the form of zinc oxide, in amounts of about 1 to 50 mg and most preferably about 15 mg. Zinc can be incorporated in the present formulation in various forms such as an oxide, a phosphate, a chloride, a sulfate, a nitrate, a gluconate, or the like, as well as metallic zinc.

Copper (cuprous oxide) is a trace mineral essential for red blood cell formation. Copper is provided in the present micronutrient formulation in the form of cupric oxide, in amounts of about 0.5 to 10 mg and most preferably about 2.0 mg. Copper can be incorporated in the present micronutrient formulation in various forms such as a sulfate, a nitrate, a chloride, a carbonate, an oxide, a hydroxide, an iodide, a glutamate, an aspartate, a citrate, or the like.

Iodine (potassium iodide) is essential for proper thyroid functioning. Iodine is provided in the present micronutrient formulation in the form of a potassium salt, wherein the iodine is present in amounts of about 0.05 to 1 mg and most preferably about 0.15 mg.

The vitamins and minerals and other nutritional aids incorporated in the micronutrient of the present invention are of food-grade, approved for use in humans (U.S. Pharmacopoeia); they may be obtained from various distributors known to one of skill in the art.

Further micronutrients, including but not limited to vitamin A, vitamin K, fatty acids (including linoleic acid, linolenic acid, and omega-3 fatty acids), phosphorous, selenium, boron, biotin, choline, inositol, chromium, molybdenum, cobalt, fluorine, manganese, nickel, potassium, or the like, may be added to the micronutrient formulation of the present invention, provided they do not interfere with the components already described.

The micronutrient formulation of the present invention preferably contains the active ingredients described above, and may contain non-active excipients such as for example fillers or binders, disintegrating agents, lubricating agents, silica flow conditioners and stabilizing agents.

Disintegrating agents are included in the present formulation to assist in the dissolution of the tablet. Disintegrating agents are well known in the art and include, but are not limited to alginic acid, carboxymethylcellulose, carboxymethylcellulose sodium, hydroxypropylcellulose (low substituted), microcrystalline cellulose, powdered cellulose, colloidal silicon dioxide, sodium croscarmellose, crospovidone, methylcellulose, polacrilin potassium, povidone, sodium alginate, sodium starch glycolate, starch, disodium disulfite, disodium edathamil, disodium edetate, disodiumethylenediaminetetraacetate (EDTA), crosslinked polyvinylpyrollidines, pregelatanized starch, carboxymethyl starch, sodium carboxymethylstarch, microcrystalline cellulose. A preferred disintegrating agent consists of sodium crosscarmellose, and is provided in the present dosage unit formulation in amounts of about 2 to 100 mg preferably about 30 to 40 mg.

Lubricating agents are included in the present formulation to assist in the compression of the formulation. Lubricating agents are well known in the art and include, but are not limited to calcium stearate, canola oil, glyceryl palmitosstearate, hydrogenated vegetable oil (type I), magnesium oxide, magnesium stearate, mineral oil, poloxamer, polyethylene glycols, sodium lauryl sulfate, sodium stearate fumarate, stearic acid, talc, zinc stearate, glyceryl behapate, magnesium lauryl sulfate, boric acid, sodium benzoate, sodium acetate, sodium benzoate/sodium acetate (in combination) and D,L-leucine. Preferred lubricants consists of magnesium stearate and sodium lauryl sulfate and are provided in the present AM multi-vitamin formulation in amounts of about 1 to 20 mg and most preferably equal amounts of about 3 to 4 mg.

Fillers or binders well known in the art, are included in the present formulation and include, but are not limited to acacia, alginic acid, calcium phosphate (dibasic), carboxymethylcellulose, carboxymethylcellulose sodium, hydroxyethylcellulose, hydroxypropylcellulose, hydroxypropylmethylcellulose, dextrin, dextrates, sucrose, tylose, pregelatinized starch, calcium sulfate, amylose, glycine, bentonite, maltose, sorbitol, ethylcellulose, disodium hydrogen phosphate, disodium phosphate, disodium pyrosulfite, polyvinyl alcohol, gelatin, glucose, guar gum, liquid glucose, compressible sugar, magnesium aluminum silicate, maltodextrin, polyethylene oxide, polymethacrylates, povidone, sodium alginate, microcrystalline cellulose, starch and zein. Preferred fillers or binders consists of microcrystalline cellulose and starch, and are provided in the present morning dosage unit in amounts of 10 to 500mg and most preferably about 180 mg and 55 mg respectively.

Many other pharmaceutically acceptable tableting agents such as fillers or binders, lubricating agents, disintegrating agents, silica flow conditioners and stabilizing agents known in the pharmaceutical arts can be used in the formulation and tableting of the micronutrient formulation of the present invention (see, e.g. Remington: The Science and Practice of Pharmacy and Handbook of Pharmaceutical Excipients; Kibbe: Handbook of Pharmaceutical Excipients). As used herein, pharmaceutically acceptable is any agent suitable for use in humans without undue side effects, such as irritation, toxicity, or allergic response.

### EXAMPLE 1

The following is an example of a morning dosage unit core formulation:

TABLE 1
Core ingredients:

| Item # | Ingredient | Label Claim | | mg/Tab. |
|---|---|---|---|---|
| 1. | Beta Carotene | 2700 | IU | |
| 2. | Vitamin E | 30 | IU | |
| 3. | Vitamin C | 120 | mg | |
| 4. | Vitamin B₁ | 3 | mg | |
| 5. | Vitamin B₂ | 3.4 | mg | |
| 6. | Vitamin B₃ | 20 | mg | |
| 7. | Vitamin B₆ | 10 | mg | |
| 8. | Pantothenic Acid | 5 | mg | |
| 9. | Magnesium | 50 | mg | |
| 10. | Iodine | 0.15 | mg | |
| 11. | Iron | 35 | mg | |
| 12. | Copper | 2 | mg | |
| 13. | Zinc | 15 | mg | |
| 14. | Cross carmellose | | | 35 |
| | Sodium | | | |
| 15. | Sodium Lauryl | | | 3.5 |
| | Sulphate | | | |
| 16. | Microcrystalline | | | 180 |
| | Cellulose PH102 | | | |
| 17. | Starch 1500 | | | 55 |
| 18. | Magnesium | | | 3.5 |
| | Stearate | | | |

The following is an example of an evening dosage unit core formulation:

TABLE 2
Core ingredients:

| Item # | Ingredient | Label Claim | | Mg/Tab. |
|---|---|---|---|---|
| 1. | Vitamin D₃ | 250 | IU | |
| 2. | Calcium | 300 | mg | |
| 3. | Vitamin B₁₂ | 12 | mcg | |
| 4. | Folic Acid | 1.1 | mg | |
| 5. | Cross carmellose | | | 30 |
| | Sodium | | | |
| 6. | Sodium Lauryl | | | 3 |
| | Sulfate | | | |
| 7. | Magnesium | | | 3 |
| | Stearate | | | |

### Dispensing Kit

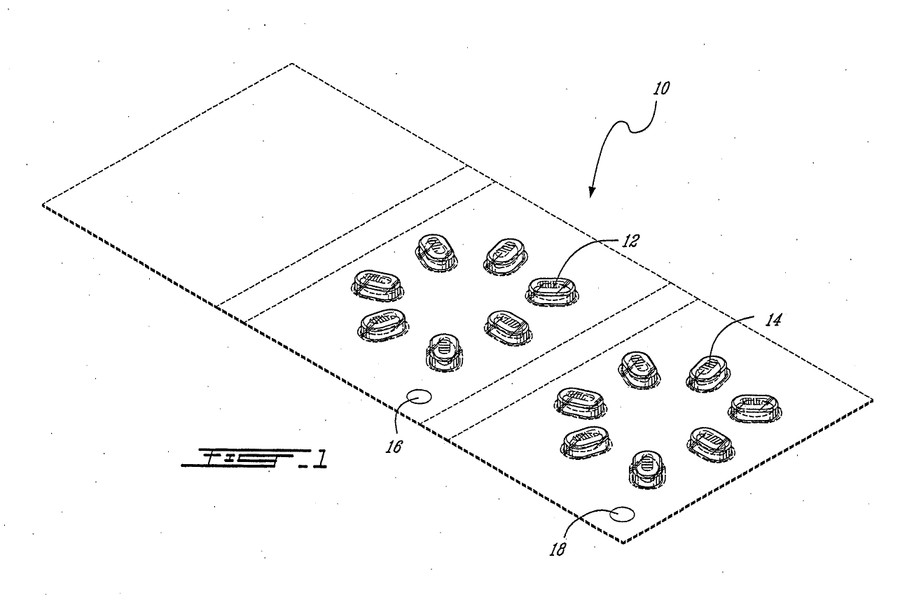

Referring now to FIG. 1, the product of the present invention may be conveniently marketed as a dispensing kit containing distinct dosage units grouped by type. Blister packs [10] of a week's worth of the supplement of the present invention having an array [12] of a first type of dosage unit to be taken at a given time of day and an array [14] of a second type of dosage unit to be taken at another time of day. Conveniently, 5 blister packs can be grouped in a box (not shown) for sale as monthly dosage packs. Advantageously, the package of dosage units will contain a 30 day supply, as four 7-day blister packs and one 2-day blister pack.

In the case of pregnant women or those wishing to become pregnant, Ideally, a woman expecting to become pregnant may start taking the multivitamin and mineral supplement of the present invention at least three months prior to pregnancy, thereafter during the entire pregnancy and during a postpartum period of at least three months.

Still referring to FIG. 1, the blister pack includes graphical means [16] and [18] permitting a patient to differentiate between the morning and evening dosage types. These means may be, for example, a color code or diagrams surrounding a particular array of dosage units of the same type be it morning or evening.

Another benefit of the blister pack is that micronutrient supplements often have an unpleasant odor. By providing a “blister pack”, each tablet is confined to its individual blister, significantly reducing odor emanations.

The cores of the formulations of the present invention are preferably coated to achieve a chosen wear resistance, aesthetic appearance, external finish or dissolution profile. Enteric, seal or color coats can be used. This may be accomplished by tablet coating procedures well known to those skilled in the pharmaceutical arts, such as for example pan coating or spray coating.

In a most preferred embodiment, the morning dosage unit of the present invention is provided with a sprayed-on Opadry Pink™ coating and polished with carnauba wax to avoid sticking. Still in a most preferred embodiment the evening dosage unit is provided with a sprayed-on Opadry Blue™ coating and also polished with carnauba wax.

It is to be understood that although a preferred embodiment of the invention is in the form of oral tablets, other dosage units and routes of administration could be used such as sublingual, rectal, intravenous, topical, etc.

Although the present invention has been described hereinabove by way of preferred embodiments thereof, it can be modified without departing from the spirit and nature of the subject invention as defined in the appended claims.

1-32. (canceled)

33. A kit for use in the treatment or prevention of micronutrient deficiencies, in women of child bearing age afflicted with or at risk to micronutrient deficiencies and who are women contemplating becoming pregnant, pregnant women or postpartum women, said kit containing the following components:
(1) from about 7 days to about a month-supply of tablets of a first type, each tablet of the first type comprising a pharmaceutically acceptable form of iron while being free of folic acid, and
(2) from about 7 days to about a month-supply of tablets of a second type, each tablet of the second type comprising a pharmaceutically acceptable form of folic acid and calcium while being free of iron, said tablets of the first and second types being of a size for ease of swallowing; and a means for having the components arranged in a way as to facilitate compliance with the regimen;
said kit being for daily use during a period from about 7 days to about a month, said means including instructions for daily intake of a tablet of the second type; and indications for a tablet of the first type to be taken daily at a predetermined time interval of at least 4 hours from the tablet of the second type and indication that, upon occurrence of side-effects the daily intake of the tablet of the first type is to be discontinued.

34. The kit of claim 33, wherein the tablets of the second type further comprise a pharmaceutically acceptable form of vitamin B₁₂.

35. The kit of claim 33, wherein the tablets of the second type further comprise a pharmaceutically acceptable form of vitamin D₃.

36. The kit of claim 33, wherein the predetermined time interval is 8 to 12 hours.

37. The kit of claim 33, wherein the tablets of the first and second types are distinguishable by color coding.

38. The kit of claim 33, wherein the tablets of the first type each comprise:
(a) from 2 to 300 mg of iron;
(b) from 250 to 5000 I.U. of beta-carotene;
(c) from 0.5 to 10 mg of vitamin B₁;
(d) from 0.5 to 10 mg of vitamin B₂;
(e) from 2 to 50 mg of vitamin B₃;
(f) from 2 to 100 mg of vitamin B₆;
(g) from 0.5 to 20 mg of pantothenic acid;
(h) from 10 to 1000 mg of vitamin C;
(i) from 1 to 500 I.U. of vitamin E;
(j) from 1 to 50 mg of zinc;
(k) from 0.5 to 10 mg of copper;
(l) from 5 to 200 mg of magnesium; and
(m) from 0.05 to 1 mg of iodine;
and the tablets of the second type each comprise:
(a) from 0.1 to 10 mg of folic acid;
(b) from 10 to 1500 mg of calcium;
(c) from 2 to 50 mcg of vitamin B₁₂; and
(d) from 10 to 1000 I.U. of vitamin D₃.

39. The supplement kit of claim 33, wherein the tablets of the first type each comprise:
(a) about 35 mg of iron, present in the form of ferrous fumarate;
(b) about 2700 I.U. of beta-carotene;
(c) about 3 mg of vitamin B₁, present in the form of thiamine mononitrate;
(d) about 3.4 mg of vitamin B₂, present in the form of riboflavin;
(e) about 20 mg of vitamin B₃, present in the form of niacinamide;
(f) about 10 mg of vitamin B₆, present in the form of pyridoxine HCl;
(g) about 5 mg of pantothenic acid, present in the form of calcium pantothenate;
(h) about 120 mg of vitamin C, present in the form of ascorbic acid;
(i) about 30 I.U. of vitamin E, present in the form of dl-α-tocopheryl acetate;
(j) about 15 mg of zinc, present in the form of zinc oxide;
(k) about 2 mg of copper, present in the form of cupric oxide;
(l) about 50 mg of magnesium, present in the form of magnesium oxide; and
(m) about 0.15 mg of iodine, present in the form of potassium iodide;
and the tablets of the second type each comprise:
(a) about 1.1 mg of folic acid;
(b) about 300 mg of a calcium, present in the form of calcium carbonate;
(c) about 12 mcg of vitamin B₁₂, present in the form of cyanocobalamine; and
(d) about 250 I.U. of vitamin D₃, present in the form of cholecalciferol.

40. A method of treating or preventing micronutrient deficiencies in a woman of child bearing age afflicted with or at risk for micronutrient deficiencies and who are in gestation, recently gave birth or are contemplating becoming pregnant with a micronutrient supplement comprising folic acid and iron, wherein the micronutrient supplement consists of two distinct types of micronutrient tablets, a first type of tablets comprising a pharmaceutically acceptable form of iron while being free of folic acid and a second type of tablets comprising a pharmaceutically acceptable form of folic acid and calcium while being free of iron, wherein the tablets of the first and second types are of a size for ease of swallowing, and wherein the micronutrient supplement is provided daily and contains a supply for about 7 days to about a month of a tablet of the first type and of the second type, both types being adapted to be administered at a predetermined time interval from each other of at least 4 hours, with a rest period from 0 day to about a month, during which a tablet of the second type only is used daily.

41. The method of claim 40, wherein the tablets of the second type further comprise a pharmaceutically acceptable form of vitamin B₁₂.

42. The method of claim 40, wherein the tablets of the second type further comprise a pharmaceutically acceptable form of vitamin D₃.

43. The method of claim 40, wherein the tablets of the first type each comprise:
(a) from 2 to 300 mg of iron;
(b) from 250 to 5000 I.U. of beta-carotene;
(c) from 0.5 to 10 mg of vitamin B₁;
(d) from 0.5 to 10 mg of vitamin B₂;
(e) from 2 to 50 mg of vitamin B₃;
(f) from 2 to 100 mg of vitamin B₆;
(g) from 0.5 to 20 mg of pantothenic acid;
(h) from 10 to 1000 mg of vitamin C;
(i) from 1 to 500 I.U. of vitamin E;
(j) from 1 to 50 mg of zinc;
(k) from 0.5 to 10 mg of copper;
(l) from 5 to 200 mg of magnesium; and
(m) from 0.05 to 1 mg of iodine;
and the tablets of the second type each comprise:
(a) from 0.1 to 10 mg of folic acid; and
(b) from 10 to 1500 mg of calcium;
(c) from 2 to 50 mcg of vitamin B₁₂;
(d) from 10 to 1000 I.U. of vitamin D₃.

44. The method of claim 40, wherein the tablets of the first type each comprise:
(a) about 35 mg of iron, present in the form of ferrous fumarate;
(b) about 2700 I.U. of beta-carotene;
(c) about 3 mg of vitamin B₁, present in the form of thiamine mononitrate;
(d) about 3.4 mg of vitamin B₂, present in the form of riboflavin;
(e) about 20 mg of vitamin B₃, present in the form of niacinamide;
(f) about 10 mg of vitamin B₆, present in the form of pyridoxine HCl;
(g) about 5 mg of pantothenic acid, present in the form of calcium pantothenate;
(h) about 120 mg of vitamin C, present in the form of ascorbic acid;
(i) about 30 I.U. of vitamin E, present in the form of dl-α-tocopheryl acetate;
(j) about 15 mg of zinc, present in the form of zinc oxide;
(k) about 2 mg of copper, present in the form of cupric oxide;
(l) about 50 mg of magnesium, present in the form of magnesium oxide; and
(m) about 0.15 mg of iodine, present in the form of potassium iodide;
and the tablets of the second type each comprise:
(a) about 1.1 mg of folic acid;
(b) about 300 mg of a calcium, present in the form of calcium carbonate;
(c) about 12 mcg of vitamin B₁₂, present in the form of cyanocobalamine; and
(d) about 250 I.U. of vitamin D₃, present in the form of cholecalciferol.

45. The method of claim 40, wherein the tablets of the first and second types are distinguishable by color coding.
